(19) United States
(12) Patent Application Publication
(10) Pub. No.: US 2009/0318960 A1
(43) Pub. Date: Dec. 24, 2009

(54) METHOD OF KNOTLESS TISSUE FIXATION WITH CRISS-CROSS SUTURE PATTERN

(76) Inventor: Stephen S. Burkhart, San Antonio, TX (US)

Correspondence Address: DICKSTEIN SHAPIRO LLP, 1825 EYE STREET NW, Washington, DC, 20006-5403, US

(21) Appl. No.: 12/488,392

(22) Filed: Jun. 19, 2009

Related U.S. Application Data
(63) Continuation-in-part of application No. 11/700,916, filed on Feb. 1, 2007.
(60) Provisional application No. 61/074,545, filed on Jun. 20, 2008.
(60) Provisional application No. 60/763,915, filed on Feb. 1, 2006.

(51) Int. Cl. A61B 17/04

(52) U.S. Cl. 606/228; 606/232

(57) ABSTRACT

A knotless tissue fixation (such as an arthroscopic rotator cuff repair) with a criss-cross suture pattern. The criss-cross pattern is obtained by (i) providing a first medial row constructed with a first plurality of fixation devices, at least one of the first plurality of fixation devices being an anchor; (ii) providing a second lateral row constructed with a second plurality of fixation devices, at least one of the second plurality of fixation devices being a knotless fixation device; and (iii) providing a structure formed of suture, suture chain, tape or allograft/biological component, and extending the structure in a criss-cross pattern, over the soft tissue, so that the structure is secured in place by the anchors.

### CROSS-REFERENCE TO RELATED APPLICATIONS

This application claims the benefit of U.S. Provisional Application No. 61/074,545, filed Jun. 20, 2008, the entire disclosure of which is incorporated by reference herein. This application is also a continuation-in-part application of U.S. patent application Ser. No. 11/700,916, filed Feb. 1, 2007, which in turn claims the benefit of U.S. Provisional Application No. 60/763,915, filed Feb. 1, 2006, the entire disclosures of which are also incorporated by reference herein.

### FIELD OF THE INVENTION

The present invention relates to surgical fixation and, more particularly, to a method of knotless tissue fixation with a criss-cross suture pattern.

### BACKGROUND OF THE INVENTION

Rotator cuff tear is one of the most common conditions causing shoulder disability in patients. Failure of the rotator cuff tendon results in pain over the anterior and lateral aspects of the shoulder and loss of shoulder function. Typically, patients exhibit weakness in elevating the arm and lose the ability to work overhead. The rotator cuff tear may be repaired using various surgical procedures.

Recently, there has been an increased interest in the re-establishment of the normal anatomy of the rotator cuff footprint using arthroscopic rotator cuff repair techniques. Several different techniques of arthroscopic repair of rotator cuff tear are currently well-known, and include single-row, double-row and SutureBridge™ techniques.

In the single-row technique, a cannula, preferably, an Expanula™ Cannula (AR-6569 sold by Arthrex, Inc., Naples, Fla.) is used to retract the deltoid and expand the working area in the subacromial space. A Double Scorpion™ suture passer (AR-13994 sold by Arthrex, Inc., Naples, Fla.) is used to pass an inverted FiberTape® mattress stitch in a single step. Tails of the FiberTape® are retrieved through the Expanula™ Cannula, loaded through an eyelet of a SwiveLock™ C suture anchor and the anchor is inserted into a prepared bone socket until the anchor body contacts bone. Single-row suture anchor techniques have been criticized because of their inability to restore the normal medial-to-lateral width of the rotator cuff footprint.

In the double-row technique, set forth in U.S. Patent Application Publication No. 2007/0135843, the disclosure of which is herein incorporated by reference, one row of anchors is placed in a medial aspect of the rotator cuff footprint and another row of anchors is placed in a lateral aspect of the footprint. First, Bio-Corkscrews® with FiberChain® are inserted in a row in the medial aspect and the free ends of the FiberChain® are passed. A second link, from the rotator cuff edge, of each of the free ends of the FiberChain® is captured with an implant having a forked tip. The forked tip implant is inserted to the bottom of the bone socket and an anchor is screwed in behind the implant to until the anchor is fully seated in bone.

In the SutureBridge™ technique, set forth in U.S. Patent Application Publication No. 2007/0191849 (U.S. patent application Ser. No. 11/700,916, filed Feb. 1, 2007), the disclosure of which is herein incorporated by reference, a medial row is constructed with two, fully threaded Corkscrew® FT anchors, combined with knotless lateral fixation using two PushLocks®. This construct provides stability in rotation and protects a broad healing zone from synovial fluid infiltration.

The anchors in the single-row, double-row and the SutureBridge™ techniques described above may be secured in the bone socket using the PushLock™ technique, the SwiveLock™ technique or other knotless suture fixation techniques. U.S. Pat. No. 7,329,272, issued on Feb. 12, 2008, the disclosure of which is incorporated by reference herein, discloses the PushLock™ technique, and U.S. Application Publication No. 2008/0004659, the disclosure of which is incorporated by reference herein, discloses the SwiveLock™ technique for knotless fixation of tissue.

Although the above-described techniques provide an improved method of rotator cuff repair, there is a need for an arthroscopic surgical technique which provides a larger area of rotator cuff footprint compression, a more even compression over the entire footprint, and better maintenance of pressure over time under cyclic loading. Accordingly, there exists a need in the art for an improved rotator cuff repair technique.

### SUMMARY OF THE INVENTION

The present invention fulfills the needs noted above by providing a knotless tissue fixation (such as an arthroscopic rotator cuff repair) with a criss-cross suture pattern. The criss-cross pattern is obtained by (i) providing a first medial row constructed with a first plurality of fixation devices, at least one of the first plurality of fixation devices being an anchor; (ii) providing a second lateral row constructed with a second plurality of fixation devices, at least one of the second plurality of fixation devices being a knotless fixation device; and (iii) providing a structure formed of flexible strands of suture, suture chain, tape or allograft/biological component, and extending the flexible strand in a criss-cross pattern, over the soft tissue, so that the flexible strand is secured in place by the anchors. The knotless tissue fixation (such as an arthroscopic rotator cuff repair) with a criss-cross suture pattern of the present invention reduces the “dog ears” (i.e., redundant tissue at the margin of the tissue to be repaired—the torn tissue) as the suture strands (or suture chain or tape, for example) cross and compress the tear margin at smaller and more equal intervals.

These and other features and advantages of the present invention will become apparent from the following description of the invention that is provided in connection with the accompanying drawings and illustrated embodiments of the invention.

### BRIEF DESCRIPTION OF THE DRAWINGS

FIGS. 1-4 illustrate an exemplary rotator cuff repair using the diamondback suture bridge technique (criss-cross pattern technique) of the present invention; and

FIG. 5 illustrates a detailed view of the criss-cross pattern of FIG. 4.

### DETAILED DESCRIPTION OF THE PREFERRED EMBODIMENTS

The present invention provides methods for conducting anatomical tissue repair, such as ligament repair and reconstruction. The present invention provides a method of knotless tissue fixation with a criss-cross suture pattern. The criss-cross pattern is obtained by (i) providing a first medial row constructed with a first plurality of fixation devices, at least one of the first plurality of fixation devices being an anchor; (ii) providing a second lateral row constructed with a second plurality of fixation devices, at least one of the second plurality of fixation devices being a knotless fixation device; and (iii) providing a structure formed of a flexible strand such as suture, suture chain, tape or an allograft/biological component, and extending the flexible strand in a criss-cross pattern, over the soft tissue, so that the flexible strand is secured in place by the anchors. The criss-cross pattern of the present invention may have an exemplary diamond shape or a diamondback bridge configuration.

The number of fixation devices in the first medial row may be similar to, or different from, the number of fixation devices in the second lateral row. Preferably, the number of fixation devices in the first medial row is different from the number of fixation devices in the second lateral row, to reduce the “dog ears” (i.e., redundant tissue at the cuff margin) as the suture strands (or suture chain or tape, for example) cross and compress the tear margin at smaller and more equal intervals. In an exemplary embodiment only, the first medial row comprises two anchors and the second lateral row comprises three push-in type anchors. Alternatively, the first medial row may comprise three anchors and the second lateral row may comprise two push-in type anchors. In other embodiments, the first medial row may comprise three anchors and the second lateral row may comprise four push-in type anchors, and so forth. Thus, the first medial row may comprise any number of fixation devices (for example, anchors) while the second lateral row may also comprise any number of fixation devices (for example, push-in anchors), which is preferably different from that of the first row anchors.

According to an exemplary and illustrative embodiment only, the patient is placed in a lateral decubitus position and held with a vacuum bean bag. Using a suture retriever/tissue grasper, the mobility of the rotator cuff tear is assessed to determine whether a U or L-shaped component exists. In the case of large tears extending to the superior aspect of the glenoid, irrespective of shape, margin convergence suturing is performed to reduce volume and strain on the repair.

Subsequent to assessing the width of the rotator cuff footprint, the most medial row is placed adjacent to the articular margin of the humerus. Two Bio-Corkscrew® FT suture anchors are placed to assure full contact of the detached tendon along the medial footprint of the greater tuberosity. Using the 45° “Deadman's” angle of optimal anchor insertion, pilot holes are prepared for SwiveLock™ or PushLock SP™ anchors at the far lateral portion of the rotator cuff footprint and greater tuberosity. Three SwiveLock™ or PushLock SP™ anchors are placed in a linear fashion from anterior to posterior to assist in creating a criss-cross diamondback compression pattern. Different color FiberChain® sutures may be used to form the criss-cross compression pattern.

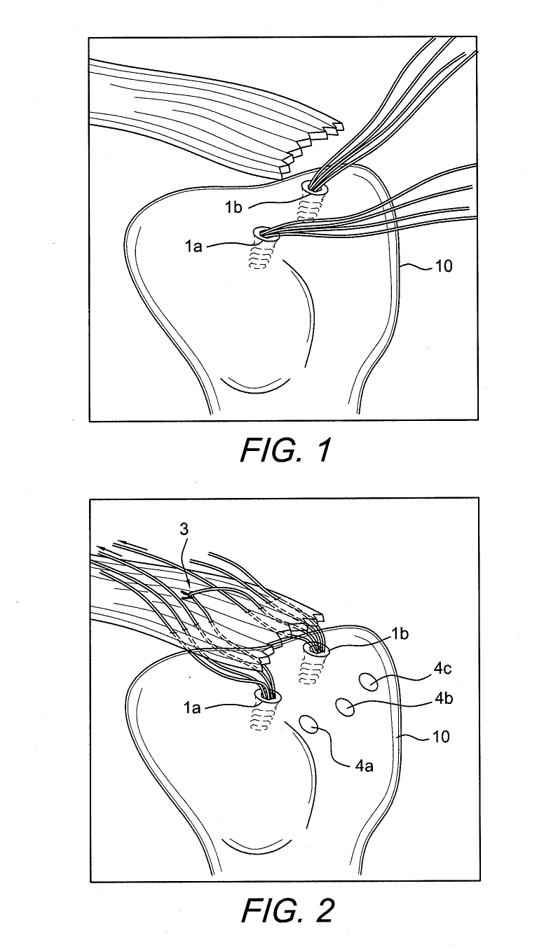

Referring now to the drawings, where like elements are designated by like reference numerals, FIGS. 1-5 illustrate an exemplary technique of the present invention. Details of the various instruments, accessories and implants used in the technique of the present invention are listed below in Table 1.

TABLE 1
List of Instruments, Accessories and Implants for the
Criss-Cross Diamond Back Compression Pattern Technique
PushLock SP ™

| | |
|---|---|
| Description: | The PushLock SP ™ was developed to help speed completion of a |
| | SutureBridge ™ while increasing the precision of the final construct. |
| | It combines a small titanium tip with either a PLLA or PEEK anchor |
| | body. The titanium tip minimizes the need to prepare a bone socket |
| | for the lateral row, where soft tissue can sometimes obscure the view. |
| | The self-punching feature helps maintain proper axial alignment of |
| | the anchor during its final insertion into the bone socket. |

Bio-Corkscrew ®

| | |
|---|---|
| Description: | A bioabsorbable PLLA suture anchor that has 14 inch pounds of |
| | insertion torque strength. The strong internal drive mechanism |
| | provides double the resistance to stripping than any other |
| | bioabsorbable suture anchor available. |

Bio-SwiveLock ™ C

| | |
|---|---|
| Description: | It's a 4.75 mm or 5.5 mm twist-in knotless anchor. This anchor |
| | functions very similar to the PushLock but with a twist-in design. |
| | This anchor is available with a bioabsorbable PLLA anchor body and |
| | PEEK eyelet. It can be used as the lateral row of the suture bridge. It |
| | can also be combined with FiberTape ®. |

Scorpion ™ Suture Passer

| | |
|---|---|
| Description: | Ergonomically designed for one-hand use, the multi-function suture |
| | passer can grasp rotator cuff tissue and retrieve a suture. |

KingFisher ® Suture Retriever/Tissue Grasper

| | |
|---|---|
| Description: | It is used for arthroscopic tissue grasping/reduction and has a self- |
| | releasing jaw lock mechanism. To lock the jaws, and securely hold |
| | the tissue, pressure is applied on the posterior aspect of the forward |
| | finger. To release the lock, and open the jaws, finger pressure is |
| | transferred to the anterior portion of the forward ring. |

FiberChain ®

| | |
|---|---|
| Description: | A single stranded #2 FiberWire ® suture strand that transitions to chain |
| | links of interwoven FiberWire ®. The FiberWire ® suture is a multi- |
| | stranded long chain ultra-high molecular weight polyethylene |
| | (UHMWPE) core with a braided jacket of polyester and UHMWPE. |

FiberTape ®

| | |
|---|---|
| Description: | It is an ultra-high strength 2 mm tape using an ultrahigh molecular |
| | weight polyethylene structure. |

First, the patient is placed in a lateral decubitus position and held with a vacuum bean bag. Using a suture retriever/tissue grasper, preferably a KingFisher™ Suture Retriever/Tissue Grasper, the mobility of the rotator cuff tear is assessed to determine whether a U or L-shaped component exists. In the case of large tears extending to the superior aspect of the glenoid, irrespective of shape, margin convergence suturing is performed to reduce the volume and strain on the repair.

Subsequent to assessing the width of the rotator cuff footprint, the most medial row is placed adjacent to the articular margin of the humerus. Two fixation devices 1a, 1b (for example, two Bio-Corkscrew® FT suture anchors 1a, 1b) are placed to assure full contact of the detached tendon along the medial footprint of the greater tuberosity, as shown in FIG. 1. The patient's age and bone quality may be used to determine the size of the fixation devices (anchors), i.e., whether to use a 3.7 mm, 5 mm, 5.5 mm or 6.5 mm. The free ends of the FiberChain® 2 are pulled to bring a knot 3 down, as shown in FIG. 2.

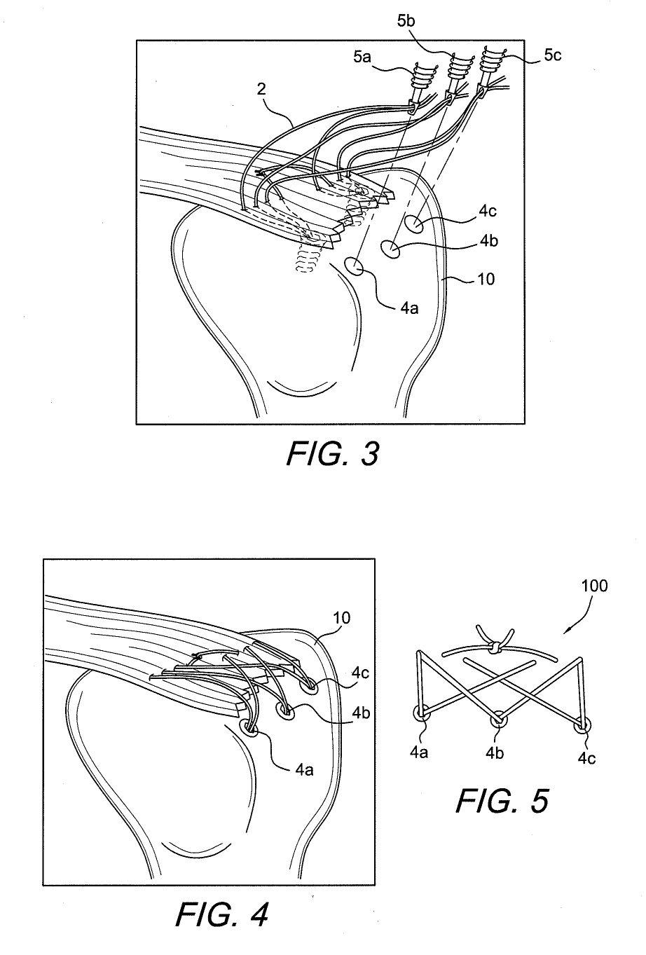

Using the 45° “Deadman's” angle of optimal anchor insertion, pilot holes 4a, 4b, 4c (FIG. 3) are prepared for insertion of the fixation devices (for example, knotless fixation devices such as SwiveLock™ or PushLock SP™ anchors) at the far lateral portion of the rotator cuff footprint and greater tuberosity. In an exemplary embodiment only, three SwiveLock™ or PushLock SP™ anchors 5a, 5b, 5c are placed in a linear fashion from anterior to posterior to assist in creating a criss-cross compression pattern, as shown in FIG. 3. Different color FiberChain® sutures may be used to form the criss-cross compression pattern. The exemplary criss-cross compression suture pattern created with the technique of the present invention is shown in FIGS. 4 and 5. The exemplary criss-cross compression suture pattern of FIG. 5 was formed with two fixation devices in the first medial row and with three fixation devices in the lateral row. However, as noted above, the criss-cross compression suture pattern of the present invention may be formed with any number of fixation devices on the medial row and any number of fixation devices on the lateral row. Preferably, the number “n” of fixation devices on the medial row is different from the number “m” of the fixation devices on the lateral row. Preferably, m=n+1, where m and n are integers, and where n≧1; or m=n−1, where m and n are integers, and where n≧2.

The technique of the present invention has several advantages over other rotator cuff repair techniques: provides a larger area of footprint compression, more even compression over the entire footprint, and better maintenance of pressure over time under cyclic loading.

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents all fall within the scope of the invention. Accordingly, the invention is not to be considered as limited by the foregoing description.

What is claimed as new and desired to be protected by Letters Patent of the United States is:

1. A knotless method of attaching soft tissue to bone, comprising:
providing a first medial row constructed with a first plurality of fixation devices, wherein at least one of the first plurality of fixation devices is an anchor;
providing a second lateral row constructed with a second plurality of fixation devices, wherein at least one of the second plurality of fixation devices is a knotless fixation device;
passing at least two flexible members from the same anchor of the first row, through the soft tissue and over a lateral portion of the soft tissue and to at least two different fixation devices of the second row, the flexible members being attached to the same anchor of the first row and being secured at an opposite end in a hole in bone by different fixation devices without tying a knot.

2. The method of claim 1, wherein the number of the fixation devices of the first row is different from the number of the fixation devices of the second row.

3. The method of claim 1, wherein the number of flexible members extending from the same anchor of the first row is equal to the number of fixation devices of the second row.

4. The method of claim 1, further comprising the formation of a structure of multiple passes of the flexible members over the soft tissue, wherein the structure is held in place by the fixation devices in the first and second rows.

5. The method of claim 4, wherein the multiple passes form a criss-cross pattern.

6. The method of claim 4, wherein the multiple passes form a diamond-shaped pattern.

7. The method of claim 4, further comprising the step of providing an implant material adjacent to the structure of multiple passes.

8. The method of claim 7, wherein the step of providing an implant material further comprises: providing aspirate bone marrow; providing a material to be implanted in the vicinity of a repair site defined by at least the structure of multiple passes; hydrating the material with aspirate bone marrow to form the implant material; and securing the implant material at the repair site.

9. The method of claim 7, wherein the implant material is selected from the group consisting of collagen, allograft and bone marrow.

10. The method of claim 7, wherein the implant material is tendon allograft impregnated with autogenous bone marrow.

11. The method of claim 1, further comprising the steps of:
providing a plurality of flexible members extending from a fixation device of the first medial row;
securing one of the plurality of flexible members to a first knotless fixation device of the second lateral row; and
securing another of the plurality of flexible members to a second knotless fixation device of the second lateral row.

12. The method of claim 11, further comprising the steps of:
providing another plurality of flexible members extending from another fixation device of the first medial row;
securing one of the plurality of flexible members to a first knotless fixation device of the second lateral row; and
securing another of the plurality of flexible members to a second knotless fixation device of the second lateral row.

13. The method of claim 1, wherein the flexible member comprises suture.

14. The method of claim 1, wherein the flexible member comprises suture tape.

15. The method of claim 1, wherein the flexible member comprises a suture chain that includes at least two loops formed of suture.

16. The method of claim 1, wherein the flexible member is comprised of an allograft or biological component.

17. The method of claim 1, wherein the soft tissue is rotator cuff.

18. A knotless method of attaching soft tissue to bone, comprising:
inserting a first anchor and a second anchor through the soft tissue, wherein each of the first and second anchors comprises a plurality of elongated flexible members secured to each of the first and second anchors prior to insertion;
inserting the first and second anchors into the bone;
passing each of the elongated flexible members over the soft tissue; and
attaching, after said step of passing, the elongated flexible members to a plurality of knotless fixation devices so that each of the elongated flexible member is secured to a different knotless fixation device, wherein the number of elongated flexible members secured to each anchor is equal to the number of knotless fixation devices.

19. The method of claim 18, wherein at least one of the knotless fixation devices is a press-in suture anchor or a swivel anchor.

20. The method of claim 18, further comprising the step of inserting the knotless fixation devices into bone, after the step of attaching the elongated flexible members.

21. The method of claim 20, wherein the step of inserting the knotless fixation devices comprises inserting the knotless fixation devices directly into the bone without the knotless fixation devices passing through the soft tissue.

22. The method of claim 18, wherein the step of attaching the elongated flexible members to the knotless fixation devices is performed without tying any knots.

23. A knotless method of attaching soft tissue to bone, comprising:
providing a first medial row constructed with two suture anchors, each of the suture anchors comprising three flexible strands attached thereto;
providing a second lateral row constructed with three knotless fixation devices;
capturing each of the three flexible strands of one of the two suture anchors of the first row by a separate knotless fixation device of the second row; and
capturing each of the three flexible strands of the other of the two suture anchors of the first row by a separate knotless fixation device of the second row.

24. The method of claim 23, wherein each of the three flexible strands is captured by one of the three knotless fixation devices.

25. The method of claim 23, further comprising the formation of a structure of multiple passes of the flexible strands over the soft tissue, wherein the structure is held in place by the anchors of the first row and the knotless fixation devices of the second row.

26. The method of claim 23, wherein at least one of the knotless fixation devices is a press-in anchor or a swivel anchor.

27. The method of claim 23, wherein the flexible strand is one of a suture, suture tape, suture chain or allograft material.

28. A method of tissue fixation comprising:
providing a plurality of suture chains, each of the suture chains including at least two loops formed of suture;
securing a first portion of the suture chains to a tissue to be fixated; and
securing a second portion of the suture chains with knotless fixation devices into bone sockets formed laterally to the tissue to be fixated, wherein the first and second portions of the suture chains form a diamond pattern.

29. The method of claim 28, further comprising the steps of:
providing a plurality of fixation devices so that each of the fixation device had a plurality of suture chains attached thereto;
passing each of the suture chains from one of the fixation devices to a separate knotless fixation device;
passing each of the suture chains from another of the fixation devices to a separate knotless fixation device; and
advancing each of the knotless fixation devices with the suture chains attached into separate bone sockets.

30. The method of claim 29, wherein the step of passing each of the suture chains to separate knotless fixation devices comprises threading the suture chain through an eyelet of the knotless fixation device.

31. The method of claim 29, wherein the knotless fixation device is a push-in type anchor with a shouldered tip.

32. The method of claim 29, wherein the knotless fixation device is one of a forked anchor, a notched anchor, a swivel anchor or a shouldered anchor.
